(19) United States
(12) Patent Application Publication
(10) Pub. No.: US 2011/0263579 A1
(43) Pub. Date: Oct. 27, 2011

(54) CHLOROTHIAZIDE, CHLOROTHIAZIDE SALTS AND PHARMACEUTICAL COMPOSITIONS THEREOF

(75) Inventors: Dhananjay Govind Sathe, Maharashtra (IN); Kamlesh Digambar Sawant, Maharashtra (IN); Tushar Anil Naik, Maharashtra (IN); Ashok Omray, Maharastra (IN); Yogendra Madhav Agashe, Maharastra (IN); Pragati Tukaram Patade, Maharastra (IN); Sunil Suresh Jog, Maharastra (IN)

(73) Assignee: USV Limited, Govandi Mumbai (IN)

(21) Appl. No.: 13/091,829

(22) Filed: Apr. 21, 2011

(30) Foreign Application Priority Data
Apr. 22, 2010 (IN) 1312/MUM/2010

(51) Int. Cl. A61K 31/549; A61P 9/12; A61P 7/10; C07D 285/32

(52) U.S. Cl. 514/223.2; 544/13

(57) ABSTRACT

The present invention relates to an improved process for the preparation of Chlorothiazide and pharmaceutically acceptable salts thereof. The present invention relates to novel polymorphs of Chlorothiazide and Chlorothiazide salts, in particular Chlorothiazide sodium. The present invention further relates to pharmaceutical compositions comprising Chlorothiazide and Chlorothiazide salts, in particular Chlorothiazide sodium and process for preparation thereof.

### RELATED APPLICATION

This application claims the benefit of Indian Provisional Application No. 1312/MUM/2010, filed on Apr. 22, 2010, Entitled: POLYMORPHS OF CHLOROTHIAZIDE SALTS AND PHARMACEUTICAL COMPOSITIONS THEREOF, which application is incorporated in its entirety.

### FIELD OF THE INVENTION

The present invention relates to an improved process for the preparation of Chlorothiazide and pharmaceutically acceptable salts thereof. The present invention relates to novel polymorphs of Chlorothiazide and Chlorothiazide salts, in particular Chlorothiazide sodium. The present invention further relates to pharmaceutical compositions comprising Chlorothiazide and Chlorothiazide salts, in particular Chlorothiazide sodium and process for preparation thereof.

### BACKGROUND OF THE INVENTION

Chlorothiazide, chemically known as 6-chloro-2H-1,2,4-benzothiadiazine-7-sulfonamide-1,1-dioxide, as represented by Formula I, has diuretic and antihypertensive activity.

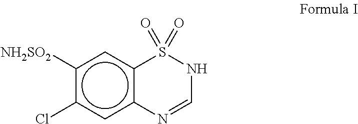

Chlorothiazide is marketed under the brand name DIURIL®, as an oral suspension.

Chlorothiazide sodium is marketed under the brand name DIURIL®, as an injectable. Chlorothiazide is also available as 250 mg and 500 mg tablets.

Chlorothiazide is a diuretic that prompts the body to eliminate excess fluid. Diuretics are an indispensable group of therapeutics used to regulate the excretion of water and salts by increasing the urinary flow. They act by diminishing sodium chloride reabsorption at different sites in the nephron thereby increasing urinary sodium chloride and water losses. The action of diuretics is based on the interference with the mechanism of ionic transport along the complete length of nephron. Chlorothiazide sodium is indicated as an adjunctive therapy in edema associated with congestive heart failure, hepatic cirrhosis and corticosteroid and estrogen therapy. It is also useful in edema caused due to various forms of renal dysfunction such as nephrotic syndrome, acute glomerulonephritis and chronic renal failure. It is indicated in the management of hypertension either as sole therapeutic agent or to enhance the effectiveness of other antihypertensive drugs in more severe forms of hypertension. The mode of action of Chlorothiazide in hypertension most probably depends upon its ability to increase sodium excretion and reduce blood volume. The ability to induce negative fluid balance has made diuretics useful in the treatment of a variety of conditions, particularly edematous states and hypertension.

U.S. Pat. No. 2,809,194 (hereinafter referred to as US '194) discloses process for preparation of Chlorothiazide (6-chloro-7-sulfamyl-1,2,4-benzothiadiazine-1,1-dioxide) comprising chlorosulfonation of m-chloroaniline to yield 5-Chloroaniline-2,4-disulfonyl chloride, which is treated with ammonium hydroxide to get 5-Chloro-2,4-disulfamylaniline. The obtained compound is treated with formic acid under reflux to get Chlorothiazide, which is crystallized using dilute alcohol to get pure desired product. The obtained Chlorothiazide can be converted into its alkali metal salt. This patent also teaches the use of ethyl orthoformate for the conversion of diamide compound to benzothiadiazine-1,1-dioxide compound.

The process disclosed in US '194 involves the use of large volume of formic acid (about 12 volumes) for cyclization step. Formic acid is irritant to skin, throat, eyes and can cause skin burns. Hence handling of such high volumes of formic acid in large scale production is not advisable.

U.S. Pat. No. 2,937,169 discloses a process for preparation of Chlorothiazide (6-chloro-7-sulfamyl-1,2,4-benzothiadiazine-1,1-dioxide) in which the ring closure is effected by admixing 5-chloro-2,4-disulfamylaniline with formic acid in an amount of one to six moles per mole of the aniline and formamide in an amount of one to six moles per mole of the aniline.

DE1126884 and ES247751 disclose a process for the preparation of Chlorothiazide where about 2 volumes of formic acid is used for the cyclization step. It was observed by the inventors of the present invention that when formic acid is used in such amounts, a thick slurry is formed which causes problems in further processing such as difficulty in stirring the reaction mass.

1514/MUM/2008 discloses a process for the preparation of Chlorothiazide or salt thereof by reacting 3-chloroaniline with chlorosulphonic acid in the presence of phosphorus trichloride followed by converting the resulting disulfonyl chloride, in the presence of ester and base, to the corresponding disulfonamide compound which is then reacted with formic acid to obtain the desired product.

1515/MUM/2008 describes the process for purification of Chlorothiazide sodium by treating Chlorothiazide with a sodium source in methanol to get Chlorothiazide sodium methanol solvate and converting the corresponding solvate into Chlorothiazide sodium.

Many organic compounds including active pharmaceutical ingredients (APIs) exhibit polymorphism. Polymorphism is the ability of a compound to exhibit more than one orientation or conformation of molecule within the crystal lattice. Various polymorphic forms of a drug substance may differ from each other in terms of stability, solubility, compressibility, flowability and spectroscopic properties thus affecting dissolution, bio-availability and handling characteristics of the substance. Rate of dissolution of an API in patient's stomach fluid can have therapeutic consequences, since it imposes an upper limit on the rate at which an orally administered API can reach the patient's bloodstream. Flowability affects the ease with which the material is handled while processing a pharmaceutical product. Knowledge of the existence of different crystal phases and their overall physical and chemical behavior is required for selection of a polymorphic form for use in the preparation of a final dosage form. To this end, investigation of crystal polymorphism is an essential step in pharmaceutical research due to the influence of solid-state properties on dosage form.

Polymorphism is not reported for Chlorothiazide and its pharmaceutically acceptable salts. Hence there is a need to study the polymorphism of Chlorothiazide and salts thereof.

The present invention provides an improved process for the preparation of Chlorothiazide or pharmaceutically acceptable salts thereof. The present invention further provides novel polymorphs of Chlorothiazide and its sodium salt. The present invention further provides pharmaceutical composition comprising Chlorothiazide/Chlorothiazide sodium or polymorphs thereof. Polymorphs of Chlorothiazide and its sodium salt prepared according to the present invention has good flow properties, especially good bulk flow properties and therefore amenable to large scale pharmaceutical processing and formulation.

### OBJECT OF THE PRESENT INVENTION

An object of the present invention is to provide an improved process for preparation of Chlorothiazide and pharmaceutically acceptable salt thereof.

Another object of the present invention is to provide a process for purification of Chlorothiazide.

Yet another object of the present invention is to provide polymorphs of Chlorothiazide, Chlorothiazide sodium and process for preparation thereof.

Yet another object of the present invention is to provide pharmaceutical composition comprising Chlorothiazide, Chlorothiazide sodium, polymorphs of Chlorothiazide/Chlorothiazide sodium and processes for preparation thereof.

Yet another object of the present invention is to provide pharmaceutical composition comprising Chlorothiazide sodium Form II, wherein the composition is in the form of an injectable and process for preparation thereof.

Yet another object of the invention is to provide pharmaceutical composition comprising Chlorothiazide sodium; wherein Chlorothiazide sodium is formed by an in situ conversion of Chlorothiazide free base to Chlorothiazide sodium and process for preparation thereof.

### SUMMARY OF THE INVENTION

The present invention discloses a process for preparation of Chlorothiazide or pharmaceutically acceptable salt thereof comprising,
a) treating 5-chloro-2,4-disulfamylaniline with about 5 volumes of formic acid to obtain Chlorothiazide;
b) optionally purifying Chlorothiazide; and
c) optionally converting Chlorothiazide to its pharmaceutically acceptable salts.
   Preferably, the treatment in step a) is carried out at a temperature of 80-100° C. for about 4 to 6 hours. Preferably, the pharmaceutically acceptable salt is Chlorothiazide sodium.

According to one aspect of the present invention, there is provided a process of purification of Chlorothiazide comprising the steps of,
a) treating Chlorothiazide with a first solvent at a temperature of about 25-100° C. or at reflux temperature to obtain a suspension;
b) optionally adding second solvent to the suspension formed in step (a); and
c) isolating purified Chlorothiazide.
   Preferably, the first solvent is selected from methanol, ethanol, isopropanol, acetone, acetonitrile, dimethyl formamide, dimethyl sulfoxide, tetrahydrofuran or 1,4-dioxane; and the second solvent is selected from water, diethyl ether, diisopropyl ether, methyl tertbutyl ether, hexane, heptane or toluene. Preferably, the isolated purified Chlorothiazide is Chlorothiazide Form I characterized by X-ray diffraction pattern having peaks at 2-theta values of about 14.42, 19.81, 20.51, 21.80, 26.29, 28.20, 28.67 and 29.10 deg.

According to another aspect of the present invention, there is provided a process wherein Chlorothiazide is treated with a polar aprotic solvent selected from dimethylformamide or dimethyl sulfoxide followed by isolation of Chlorothiazide form II characterized by X-ray diffraction pattern having peaks at 2-theta values of about 8.13, 17.02, 18.55, 19.38, 20.62, 20.95, 21.96, 26.03, 27.76 and 32.90 deg.

According to another aspect of the present invention, Chlorothiazide is converted to Chlorothiazide sodium by a process comprising the steps of,
a) treating Chlorothiazide with a suitable alkaline medium to get a solution;
b) optionally, adding antisolvent to the solution obtained in step (a); and
c) isolating Chlorothiazide sodium.
   Preferably suitable alkaline medium is selected from sodium hydroxide, sodium methoxide or sodium ethoxide in a suitable solvent selected from water, methanol, ethanol, n-propanol, isopropanol or mixture thereof; and antisolvent is selected from hexane, heptane, toluene, dichloromethane, diethyl ether, diisopropyl ether, methyl tertbutyl ether, tetrahydrofuran, 1,4-dioxane, acetonitrile, acetone, ethyl methyl ketone, 2-butanone or mixture thereof.

Another aspect of the present invention provides Chlorothiazide sodium selected from the group consisting of substantially pure Chlorothiazide sodium Form I characterized by X-ray diffraction pattern as shown in FIG. 5; or Chlorothiazide sodium Form II characterized by X-ray diffraction pattern having peaks at 2-theta values of about 17.22, 19.82, 25.91, 27.60, 29.55 and 31.21 deg; or Chlorothiazide sodium Form III characterized by X-ray diffraction pattern having peaks at 2-theta values of about 8.83, 10.41, 16.70, 18.15, 20.62, 23.31, 26.38, 26.59 and 28.11 deg; or Chlorothiazide sodium Form IV characterized by X-ray diffraction pattern having peaks at 2-theta values of about 16.55, 17.12, 17.56, 18.09, 19.55, 19.72 and 27.52 deg.

Yet another aspect of the present invention provides a compound selected from the group consisting of:
a) Chlorothiazide Form II characterized by X-ray diffraction pattern having peaks at 2-theta values of about 8.13, 17.02, 18.55, 19.38, 20.62, 20.95, 21.96, 26.03, 27.76 and 32.90 deg.;
b) Chlorothiazide sodium Form II characterized by X-ray diffraction pattern having peaks at 2-theta values of about 17.22, 19.82, 25.91, 27.60, 29.55 and 31.21 deg.;
c) Chlorothiazide sodium Form III characterized by X-ray diffraction pattern having peaks at 2-theta values of about 8.83, 10.41, 16.70, 18.15, 20.62, 23.31, 26.38, 26.59 and 28.11 deg; and
d) Chlorothiazide sodium Form IV characterized by X-ray diffraction pattern having peaks at 2-theta values of about 16.55, 17.12, 17.56, 18.09, 19.55, 19.72 and 27.52 deg.

Another aspect of the present invention provides a pharmaceutical composition comprising,
a) a therapeutically effective amount of Chlorothiazide, Chlorothiazide sodium or polymorphic form prepared by the process as described herein;
b) a pharmaceutically acceptable tonicity agent; and
c) a pharmaceutically acceptable pH adjusting agent.

Another aspect of the present invention provides a pharmaceutical composition of Chlorothiazide sodium wherein Chlorothiazide sodium is formed by an in situ conversion of Chlorothiazide free base to Chlorothiazide sodium.

Preferably Chlorothiazide used in the pharmaceutical composition is Chlorothiazide Form I; Chlorothiazide sodium used in the pharmaceutical composition is Chlorothiazide sodium Form II; pH adjusting agent is selected from the group consisting of sodium hydroxide, potassium hydroxide, alkali or alkaline earth metal carbonates/bicarbonates or mixture thereof; and tonicity agent is selected from sodium chloride, dextrose, mannitol, sucrose, lactose, galactose, trehalose, glycine or mixture thereof.

Another aspect of the present invention provides pharmaceutical composition of Chlorothiazide sodium prepared by a process comprising the steps of,
1. preparing a solution by adding pH adjusting agent in a pharmaceutically acceptable vehicle;
2. adding and dissolving Chlorothiazide sodium or polymorphs thereof, into the solution of step (a) to obtain Solution A;
3. adding and dissolving tonicity agent into said Solution A to obtain Solution B;
4. optionally adjusting the pH of said Solution B between 9.0 and 10.0 by adding a pH adjusting agent to obtain Solution C;
5. optionally adjusting the volume of said Solution B or Solution C by adding additional pharmaceutically acceptable vehicle;
6. filtering said Solution B or Solution C or solution from step e) through a membrane filter;
7. filling said filtered solution into a vial; and
8. optionally lyophilizing said solution from step g) to obtain a white lyophilized product.

Another aspect of the present invention provides a process for preparation of parenteral composition comprising Chlorothiazide sodium, said process comprising:
1. preparing a dispersion of Chlorothiazide free base Form I in a pharmaceutically acceptable vehicle;
2. preparing a solution of sodium hydroxide in a pharmaceutically acceptable vehicle;
3. converting in situ said Chlorothiazide free base Form I into Chlorothiazide sodium by addition of sodium hydroxide solution into Chlorothiazide dispersion of step a) to obtain Solution A;
4. adding and dissolving the tonicity agent into said Solution A to obtain Solution B;
5. optionally adjusting the pH of said Solution B between 9.0 and 10.0 by adding a pH adjusting agent to obtain Solution C;
6. optionally adjusting the volume of said Solution B or Solution C by adding additional pharmaceutically acceptable vehicle;
7. filtering said Solution B or Solution C or solution from step f) through a membrane filter;
8. filling said filtered solution into a vial; and
9. optionally lyophilizing said solution from step h) to obtain a white lyophilized product.

According to a preferred aspect, the addition of sodium hydroxide solution into Chlorothiazide dispersion is carried out either by slow addition method or fast addition method.

### BRIEF DESCRIPTION OF THE DRAWINGS

FIG. 1: X-ray diffraction pattern of Chlorothiazide Form I.

FIG. 2: X-ray diffraction pattern of Chlorothiazide Form II.

FIG. 3: X-ray diffraction pattern of Chlorothiazide sodium, present in the marketed Diuril sodium (chlorothiazide sodium) injection, lyophilized powder (Lot D09015).

FIG. 4: X-ray diffraction pattern of Chlorothiazide Sodium Form I obtained according to U.S. Pat. No. 2,809,194.

FIG. 5: X-ray diffraction pattern of substantially pure Chlorothiazide sodium Form I.

FIG. 6: X-ray diffraction pattern of Chlorothiazide sodium Form II.

FIG. 7: X-ray diffraction pattern of Chlorothiazide sodium Form III.

FIG. 8: X-ray diffraction pattern of Chlorothiazide sodium Form IV.

### DETAILED DESCRIPTION OF THE INVENTION

The present invention provides an improved process for the preparation of Chlorothiazide or pharmaceutically acceptable salt thereof, particularly Chlorothiazide sodium.

According to one embodiment of the present invention, process for preparation of Chlorothiazide comprises treating 5-chloro-2,4-disulfamylaniline with about 98% to 100% formic acid at temperature of about 80-100° C., preferably 90-100° C. to get a suspension. The suspension is maintained at 90-95° C. for about 4 to 6 hours at which the suspension starts becoming clear. At the same temperature, the product precipitates out from the clear solution which is then isolated and purified.

The above process is represented in Scheme I below,

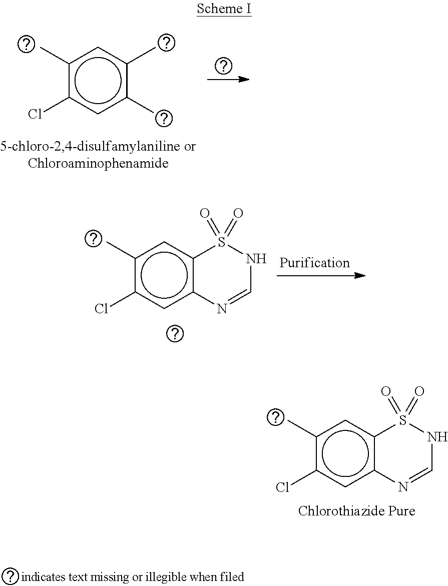

5-Chloro-2,4-disulfamylaniline is prepared from m-Chloroaniline as shown in Scheme II below,

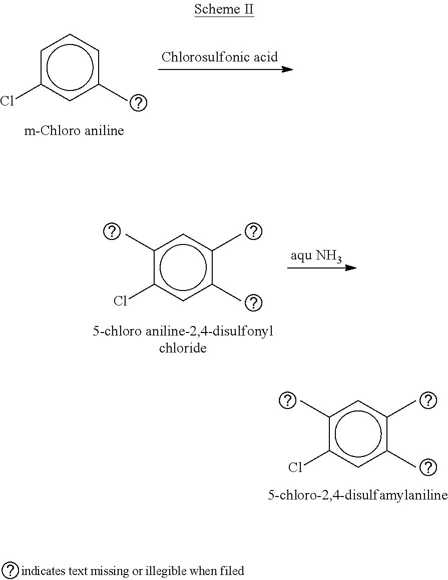

In a preferred embodiment of the present invention, process for preparation of Chlorothiazide comprises treating 5-chloro-2,4-disulfamylaniline with about 5 volumes (w.r.t disulfamylaniline) of 98% to 100% formic acid at temperature of about 90-100° C. to get a suspension. The suspension is maintained at 90-95° C. for 5 hours at which the suspension starts becoming clear. At the same temperature, the product precipitates out from the clear solution which is then isolated and purified.

The process disclosed in U.S. Pat. No. 2,809,194 uses large amount of formic acid (about 12 volumes) for cyclizing 5-chloro-2,4-disulfamylaniline to get Chlorothiazide. Thus the prior art process is not suitable on a commercial scale. DE1126884 and ES247751 discloses a process for the preparation of Chlorothiazide where about 2 volumes of formic acid is used for the cyclization step. It was observed by the inventors of the present invention that when formic acid is used in an amount of less than 5 volumes, a thick slurry is formed which causes problems in further processing such as difficulty in stirring the reaction mass. The process of the present invention provides cyclization reaction using formic acid in an amount of about 5 volumes which is very less as compared to that used in US '194 thereby making the present process economically and commercially viable. Besides, the use of about 5 volumes of formic acid helps in overcoming the problems associated with process of the prior art where 2 volumes of formic acid is used for the cyclization step.

Another embodiment of the present invention provides process for purification of Chlorothiazide comprising,
a) treating Chlorothiazide with a first solvent at a temperature of about 25-100° C. or at reflux temperature to obtain a suspension;
b) optionally, adding second solvent to the suspension formed in step a); and
c) isolating purified Chlorothiazide.

According to a preferred embodiment, Chlorothiazide is suspended in a suitable solvent and the obtained suspension is heated to a temperature in the range of about 25-100° C. preferably at temperature of about 25-80° C. or at reflux temperature for several hours followed by addition of second solvent to get clear solution. The second solvent should be miscible with the first solvent. The obtained clear solution is cooled to a temperature of about 0 to −5° C. Optionally, second solvent is added for precipitating out pure Chlorothiazide.

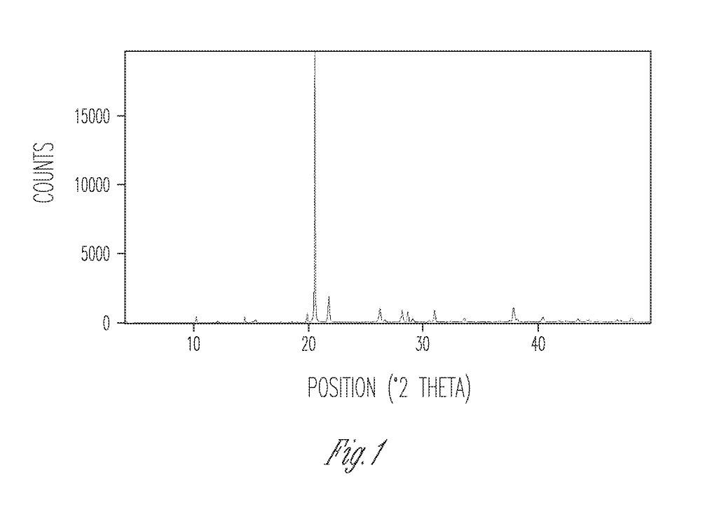

Chlorothiazide thus obtained is designated herein as Form I and characterized by X-ray diffraction pattern as shown in FIG. 1. Chlorothiazide Form I is characterized by X-ray diffraction pattern having peaks expressed as 2-theta at about 14.42, 19.81, 20.51, 21.80, 26.29, 28.20, 28.67 and 29.10 deg. Chlorothiazide Form I is further characterized by X-ray diffraction pattern having peaks expressed as 2-theta at about 10.20, 12.05, 15.33, 17.54, 25.15, 26.71, 29.67, 30.52, 31.01, 32.33, 33.59, 36.67, 37.81, 38.16, 39.16, 40.40, 41.78, 42.44, 43.47, 44.32, 45.42, 46.94, 47.28 and 48.20 deg.

The first solvent is selected from polar protic solvents such as methanol, ethanol, isopropanol or polar aprotic solvents like acetone, acetonitrile, dimethyl formamide (DMF), dimethyl sulfoxide (DMSO), tetrahydrofuran (THF) or 1,4-Dioxane preferably acetone. The second solvent is selected based on its miscibility with first solvent. In some embodiments, dissolution is obtained at reflux temperature of the solvent. The dissolution is carried out at a temperature of about 25-80° C. or at reflux temperature after addition of second solvent. The second solvent is selected from water, ether or hydrocarbon. Ether is selected from diethyl ether, diisopropyl ether, methyl tertbutyl ether (MTBE), preferably methyl tertbutyl ether. Hydrocarbon is selected from hexane, heptane or toluene, preferably toluene. The preferred second solvent is water.

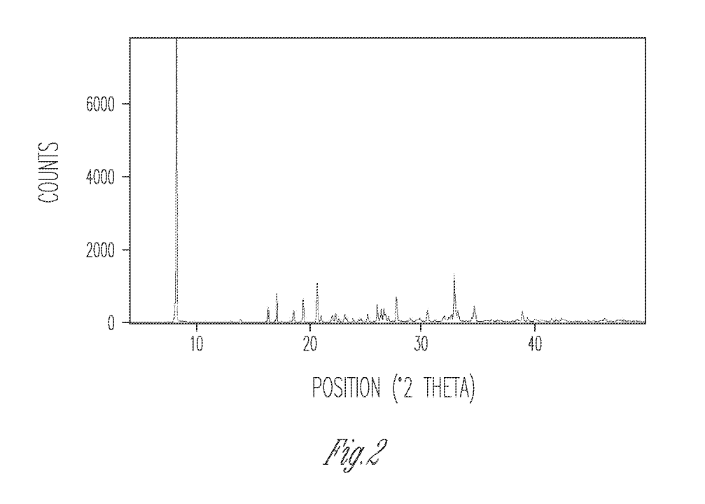

Another embodiment of the present invention provides Chlorothiazide Form II characterized by X-ray diffraction pattern as shown in FIG. 2. Chlorothiazide Form II is characterized by X-ray diffraction pattern having characteristic peaks expressed as 2-theta at about 8.13, 17.02, 18.55, 19.38, 20.62, 20.95, 21.96, 26.03, 27.76 and 32.90 deg. Chlorothiazide Form II is further characterized by peaks expressed as 2-theta at about 13.07, 22.27, 22.55, 23.80, 24.52, 25.14, 26.62, 27.02, 28.97, 29.86, 30.54, 31.18, 31.95, 32.62 and 33.24 deg.

Another embodiment of the present invention provides process for the preparation of Chlorothiazide Form II comprising the steps of:
a) dissolving Chlorothiazide in suitable solvent to obtain a solution;
b) isolating Chlorothiazide Form II.

In a preferred embodiment, dissolution is carried out at a temperature of about 25-100° C. preferably at ambient temperature. The solution thus obtained is optionally filtered to remove insoluble impurities. The solution is concentrated under vacuum at about 80-100° C. to get Chlorothiazide Form II.

Suitable solvent is selected from polar aprotic solvents such as dimethyl formamide (DMF), dimethyl sulfoxide (DMSO) or the like, preferably DMF.

U.S. Pat. No. 2,809,194 also describes conversion of Chlorothiazide to its alkali metal salts, particularly sodium salt, by dissolving Chlorothiazide in alcoholic sodium hydroxide and removing the solvent under vacuum to get sodium salt of Chlorothiazide represented as Formula II. Chlorothiazide is unstable in the presence of alkali. This leads to formation of impurities during the conversion of Chlorothiazide to Chlorothiazide sodium. These impurities will remain as a part of the final product if the product is isolated by complete removal of the solvent under vacuum as taught in US '194. The process of the present invention involves the isolation of the product by precipitation from the reaction medium thereby overcoming the problem associated with the prior art process.

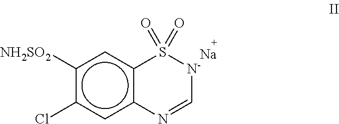

Chlorothiazide sodium exists in other polymorphic forms which are characterized and described herein.

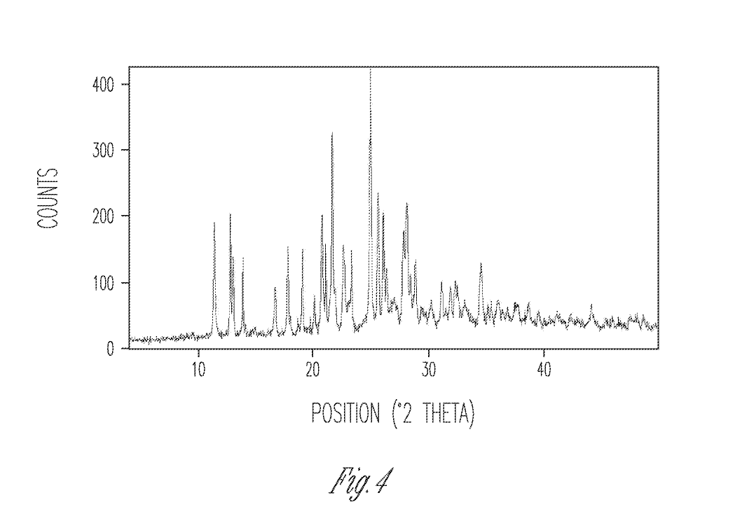

Chlorothiazide sodium obtained by following the process disclosed in U.S. Pat. No. 2,809,194 is designated herein as Chlorothiazide sodium Form I and characterized by X-ray diffraction pattern as shown in FIG. 4.

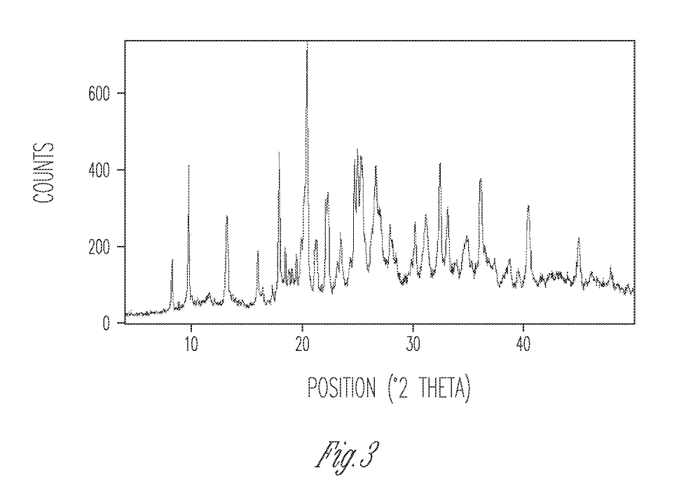

Our inventors have tested the polymorphic form of Chlorothiazide sodium present in the lyophilized formulation (B. No. Lot D09015). Chlorothiazide sodium present in the lyophilized formulation is characterized by a X-ray diffraction pattern as shown in FIG. 3.

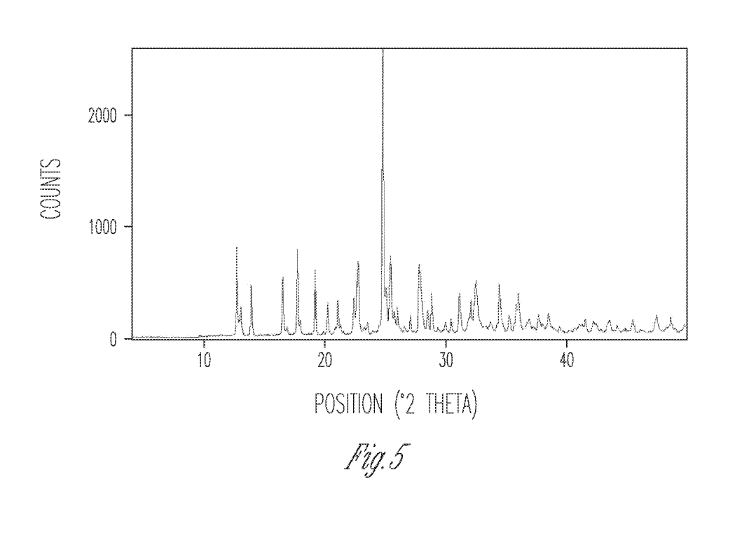

Another embodiment of the present invention provides substantially pure Chlorothiazide sodium Form I characterized by X-ray diffraction pattern as shown in FIG. 5.

Another embodiment of the present invention provides a process for preparation of substantially pure Chlorothiazide sodium Form I comprising:
a) treating Chlorothiazide with a suitable alkaline medium to get a solution;
b) optionally adding an antisolvent to the solution obtained in step a); and
c) isolating substantially pure Chlorothiazide sodium Form I.

The treatment of Chlorothiazide with a suitable alkaline medium is carried out at a temperature of about 25-100° C. or at reflux temperature of the solvent selected for dissolution. The solution is optionally cooled before/after addition of antisolvent.

In a preferred embodiment, Chlorothiazide is dissolved in an alkaline medium preferably in a methanolic solution of sodium hydroxide at 25-100° C. or at reflux temperature. The solution is optionally cooled to 5-10° C. followed by addition of an antisolvent to obtain substantially pure Chlorothiazide sodium Form I.

The alkaline medium is selected from sodium hydroxide or sodium alkoxide in a suitable solvent. Suitable solvent is selected from water or polar protic solvents such as methanol, ethanol, n-propanol, isopropanol or mixture thereof, preferably methanol. Antisolvent is selected from hydrocarbon, chlorinated hydrocarbon, ether, nitrile or ketone. Hydrocarbon is selected from hexane, heptane or toluene, preferably toluene; chlorinated hydrocarbon is methylene dichloride (MDC); ether is selected from diethyl ether, diisopropyl ether, methyl tertbutyl ether, tetrahydrofuran or 1,4-dioxane, preferably methyl tertbutyl ether; nitrile used is acetonitrile; and ketone is selected from acetone, ethyl methyl ketone or 2-butanone.

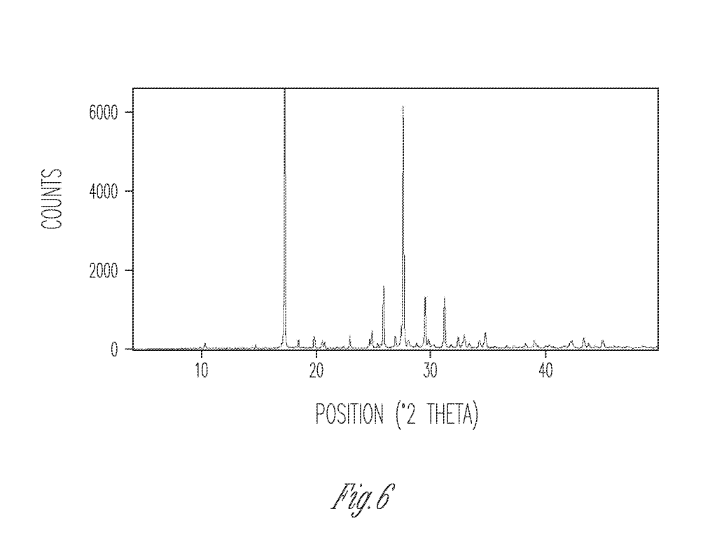

Another embodiment of the present invention provides Chlorothiazide sodium Form II characterized by X-ray diffraction pattern as shown in FIG. 6. Chlorothiazide sodium Form II is characterized by X-ray diffraction pattern having characteristic peaks expressed as 2-theta at about 17.22, 19.82, 25.91, 27.60, 29.55 and 31.21 deg. Chlorothiazide sodium Form II is further characterized by peaks expressed as 2-theta at about 10.34, 12.85, 17.88, 18.41, 20.50, 21.80, 22.96, 24.92, 26.97, 28.13, 29.86, 30.33, 31.83, 32.45 and 32.93 deg.

According to another embodiment, the present invention provides a process for preparation of Chlorothiazide sodium Form II comprising:
a) treating Chlorothiazide with a suitable alkaline medium to obtain a suspension;
b) optionally adding an antisolvent to the suspension of step a); and
c) isolating Chlorothiazide sodium Form II.

In a preferred embodiment, Chlorothiazide is suspended in an alcoholic solution preferably ethanolic solution of sodium hydroxide or sodium methoxide at 25-70° C. or at the reflux temperature of the solvent to obtain a solution. The obtained solution is cooled to 25-30° C. and stirred for 4-5 hrs maintaining the same temperature. The solution is filtered and dried immediately under vacuum at 50-70° C. to get Chlorothiazide sodium Form II.

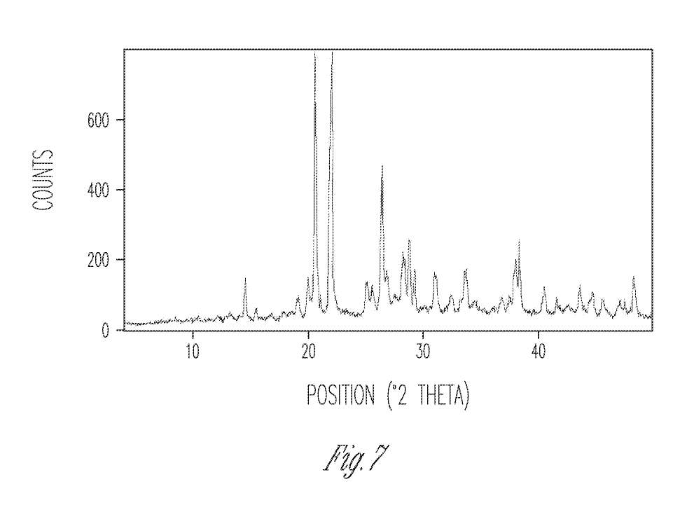

Another embodiment of the present invention provides Chlorothiazide sodium Form III characterized by X-ray diffraction pattern as shown in FIG. 7. Chlorothiazide sodium Form III is characterized by X-ray diffraction pattern having characteristic peaks expressed as 2-theta at about 8.83, 10.41, 16.70, 18.15, 20.62, 23.31, 26.38, 26.59 and 28.11 deg. Chlorothiazide sodium Form III is further characterized by peaks expressed as 2-theta at about 9.69, 11.35, 12.06, 13.47, 17.68, 19.24, 21.65, 22.17, 22.98, 25.00, 28.81, 29.21, 30.58, 31.14, 32.48, 33.72 and 34.75 deg.

According to another embodiment of the present invention there is provided a process for preparation of Chlorothiazide sodium Form III comprising
a) treating Chlorothiazide with a suitable alkaline medium to obtain a solution;
b) isolating Chlorothiazide sodium Form III.

Preferably, Chlorothiazide is treated with an alcoholic solution, preferably methanolic solution of sodium methoxide at a temperature of about 25-65° C., preferably at 25-35° C. to obtain a solution. The obtained solution is stirred for 4-5 hrs at ambient temperature to get Chlorothiazide sodium Form III.

In the practice of the process of the present invention, the alkaline medium is selected from sodium hydroxide, sodium ethoxide, sodium methoxide in a suitable solvent. Suitable solvent is selected from water, aliphatic alcohol such as methanol, ethanol, n-propanol, isopropanol or mixture thereof.

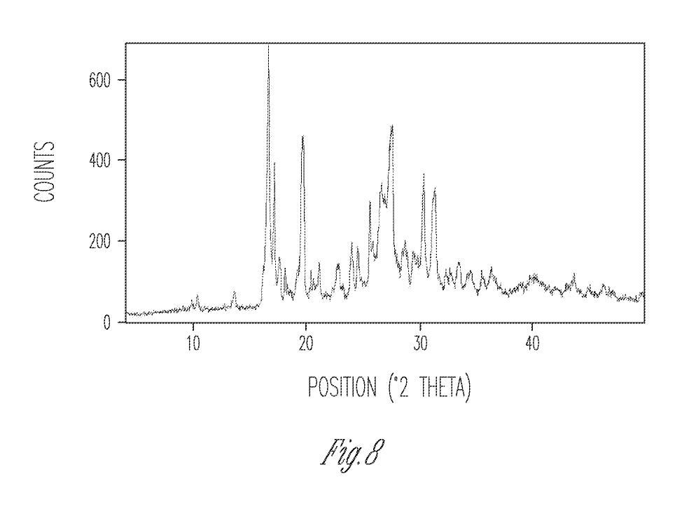

According to yet another embodiment, the present invention provides Chlorothiazide sodium Form IV characterized by X-ray diffraction pattern as shown in FIG. 8. Chlorothiazide sodium Form IV is characterized by X-ray diffraction pattern having characteristic peaks expressed as 2-theta at about 16.55, 17.12, 17.56, 18.09, 19.55, 19.72 and 27.52 deg. Chlorothiazide sodium Form IV is further characterized by peaks expressed as 2-theta at about 13.56, 21.14, 22.78, 23.98, 24.50, 25.57, 26.51, 28.69, 30.32, 31.33, 33.49, 35.57, 36.40 and 43.64 deg.

According to another embodiment, the present invention provides a process for preparation of Chlorothiazide sodium Form IV comprising drying Chlorothiazide sodium to get Chlorothiazide sodium Form IV.

Chlorothiazide sodium used for preparation of Chlorothiazide Form IV may be any polymorphic form selected from Form I, Form II, Form III or amorphous form. The drying is performed at a temperature of about 100 to 120° C. for 8-12 hrs.

The term “substantially pure” as used herein means a polymorphic form of Chlorothiazide or Chlorothiazide sodium having less than 5%, preferably less than 1%, more preferably less than 0.5% of other polymorphic forms of Chlorothiazide or Chlorothiazide sodium respectively.

Chlorothiazide used for preparation of polymorphs of Chlorothiazide or sodium salt thereof may be selected from Form I, Form II or any other form.

Chlorothiazide or Chlorothiazide sodium obtained by the process of the present invention has particle size distribution such that about 90% particles have a particle size less than about 500 microns, preferably less than about 400 microns, more preferably less than about 200 microns. The particles can be micronized to get particle size distribution such that 90% particles have particle size less than about 100 microns, preferably less than about 50 microns, more preferably less than about 10 microns.

Chlorothiazide or Chlorothiazide sodium obtained by the process of the present invention is more than about 99% pure, preferably more than about 99.5% pure with known impurities less than about 0.15% and unknown impurities less than about 0.1%.

According to one embodiment, the present invention provides pharmaceutical composition comprising Chlorothiazide or pharmaceutically acceptable salt thereof, in particular Chlorothiazide sodium or polymorphs of Chlorothiazide/Chlorothiazide sodium.

Pharmaceutical dosage forms comprising Chlorothiazide, Chlorothiazide sodium or polymorphs thereof as described herein may be used for oral as well as parenteral administration. Oral dosage form may be in the form of tablets, capsules or granules. Parenteral dosage forms may be in the form of solution or as a lyophilized product.

According to one embodiment, the present invention provides parenteral pharmaceutical composition comprising a therapeutically effective amount of Chlorothiazide sodium, preferably Chlorothiazide sodium Form II, a tonicity agent and a pH adjusting agent. Said parenteral compositions are useful as an adjunctive therapy in edema associated with congestive heart failure, hepatic cirrhosis, corticosteroid and estrogen therapy.

According to another embodiment, the invention provides parenteral pharmaceutical compositions comprising a therapeutically effective amount of Chlorothiazide base, tonicity agent and pH adjusting agent. In a preferred embodiment, Chlorothiazide used is Chlorothiazide Form I.

The pharmaceutical composition is in the form of an injectable which may be in the form of a parenteral solution which may optionally be lyophilized. Chlorothiazide sodium injection according to the invention may be in the form of solution or in the form of lyophilized product which can be reconstituted prior to administration. Said compositions are suitable for single dose intravenous administration.

According to one embodiment, Chlorothiazide sodium injection is provided as a lyophilized product which can be reconstituted by dissolving the lyophilized product in a suitable solvent such as physiological saline, aqueous solution of 5% Dextrose or distilled water for injection. The invention thus encompasses Chlorothiazide sodium for injection obtained by reconstitution of the lyophilized products as described herein.

According to a preferred embodiment, the lyophilized formulation according to the invention further comprises at least one tonicity agent selected from sodium chloride, dextrose, mannitol, sucrose, lactose, galactose, trehalose, glycine or mixture thereof, preferably mannitol. Preferably, the tonicity agent is present in an amount from about 150 mg to 350 mg.

According to another embodiment, the invention provides parenteral pharmaceutical compositions comprising Chlorothiazide sodium or polymorphs thereof, wherein Chlorothiazide sodium is formed by an in situ conversion of Chlorothiazide free base to Chlorothiazide sodium.

According to one embodiment, the invention provides pharmaceutical compositions comprising Chlorothiazide sodium in the form of an injectable dosage form comprising:
a) a therapeutically effective amount of Chlorothiazide free base;
b) at least one tonicity agent; and
c) sodium hydroxide in an amount sufficient to maintain the pH of the composition between 9.0 and 10.0;
   wherein Chlorothiazide sodium is formed by an in situ conversion of Chlorothiazide free base to Chlorothiazide sodium.

According to a preferred embodiment, the invention provides pharmaceutical compositions comprising Chlorothiazide sodium in the form of an injectable dosage form comprising:
a) a therapeutically effective amount of Chlorothiazide free base;
b) mannitol or sucrose; and
c) sodium hydroxide in an amount sufficient to maintain the pH of the composition between 9.0 and 10.0;
   wherein Chlorothiazide sodium is formed by an in situ conversion of Chlorothiazide free base to Chlorothiazide sodium.

Preferably, the tonicity agent is mannitol.

According to one embodiment, the invention provides lyophilized product comprising from about 220 mg to about 660 mg of Chlorothiazide sodium, which is equivalent to from about 200 mg to about 600 mg of Chlorothiazide. According to a preferred embodiment, the invention provides lyophilized product comprising from about 275 mg to about 550 mg of Chlorothiazide sodium, which is equivalent to from about 250 mg to about 500 mg of Chlorothiazide.

According to one embodiment, the invention provides pharmaceutical compositions in the form of an injectable dosage form comprising:
a) a therapeutically effective amount of Chlorothiazide sodium;
b) a pharmaceutically acceptable tonicity agent; and
c) a pharmaceutically acceptable pH adjusting agent in an amount sufficient to maintain the pH of the composition between 9.0 and 10.0.

Chlorothiazide sodium may be used in the range of about 1% to 20% by weight of total composition. Tonicity agents may be used in the range of about 1% to 15% by weight of total composition. pH adjusting agents may be used in the range of about 0.1% to 5% by weight of total composition.

According to preferred embodiment, the invention provides pharmaceutical compositions in the form of an injectable dosage form comprising:
a) a therapeutically effective amount of Chlorothiazide sodium;
b) mannitol or sucrose; and
c) sodium hydroxide or potassium hydroxide in an amount sufficient to maintain the pH of the composition between 9.0 and 10.0.

According to one embodiment, the invention provides pharmaceutical composition in the form of an injectable dosage form comprising:
a) a therapeutically effective amount of Chlorothiazide sodium or polymorphic form thereof;
b) a pharmaceutically acceptable tonicity agent; and
c) a pharmaceutically acceptable pH adjusting agent;
   wherein Chlorothiazide sodium is present in an amount of approximately 275 mg to 550 mg.

In a preferred embodiment, Chlorothiazide sodium is present as Chlorothiazide sodium Form II.

According to one embodiment, the invention provides pharmaceutical compositions comprising Chlorothiazide sodium in the form of an injectable dosage form comprising:
a) a therapeutically effective amount of Chlorothiazide free base;
b) at least one tonicity agent; and
c) sodium hydroxide in an amount sufficient to maintain the pH of the composition between 9.0 and 10.0;
   wherein Chlorothiazide sodium is formed by an in situ conversion of Chlorothiazide free base to Chlorothiazide sodium.

According to preferred embodiment, the invention provides pharmaceutical compositions comprising Chlorothiazide sodium in the form of an injectable dosage form comprising:
a) a therapeutically effective amount of Chlorothiazide free base;
b) mannitol or sucrose; and
c) sodium hydroxide in an amount sufficient to maintain the pH of the composition between 9.0 and 10.0;
   wherein Chlorothiazide sodium is formed by an in situ conversion of Chlorothiazide free base to Chlorothiazide sodium.

Preferably tonicity agent is mannitol.

According to one embodiment, the present invention provides a process for the preparation of a lyophilized product comprising Chlorothiazide or a pharmaceutically acceptable salt thereof, in particular Chlorothiazide sodium, comprises,
a) preparing an aqueous solution comprising a therapeutically effective amount of Chlorothiazide or pharmaceutically acceptable salt thereof, a tonicity agent and a pH adjusting agent; and
b) lyophilization of the aqueous solution obtained in step (a).
   According to one embodiment, the invention provides a process for preparing parenteral pharmaceutical composition comprising Chlorothiazide sodium, said process comprising:
a) preparing a solution by adding pH adjusting agent in a pharmaceutically acceptable vehicle;
b) adding and dissolving Chlorothiazide sodium or polymorphs thereof, into the solution of step (a) to obtain Solution A;
c) adding and dissolving tonicity agent into said Solution A to obtain Solution B;
d) optionally adjusting the pH of Solution B between 9.0 and 10.0 by adding a pH adjusting agent to obtain Solution C;
e) optionally adjusting the volume of said Solution B or Solution C by adding additional pharmaceutically acceptable vehicle;
f) filtering said Solution B or Solution C or solution from step e) through a membrane filter;
g) filling said filtered solution into a vial; and
h) optionally lyophilizing said solution from step g) to obtain a white lyophilized product.

According to a preferred embodiment, the invention provides a process for preparing a pharmaceutical composition in the form of an injectable dosage form, comprising:
a) preparing a solution by adding approximately 25 mg to 45 mg of pH adjusting agent in pharmaceutically acceptable vehicle;
b) adding and dissolving approximately 275 mg to 550 mg of Chlorothiazide sodium or polymorphs thereof, into the solution of step a) to obtain Solution A;
c) adding and dissolving approximately 150 mg to 350 mg of tonicity adjusting agent into Solution A to obtain Solution B;
d) optionally adjusting the pH of Solution B between 9.0 and 10.0 by adding a pH adjusting agent to obtain Solution C;
e) optionally adjusting the volume of the Solution B or Solution C by adding additional pharmaceutically acceptable vehicle;
f) filtering the Solution B or Solution C or solution from step e) through a membrane filter;
g) filling said filtered solution from step f) into a vial; and
h) optionally lyophilizing said solution from step g) to obtain a white lyophilized product.

Optionally, nitrogen sparging can be used during the formulation as the use of a nitrogen sparge during formulation compounding can reduce dissolved oxygen substantially.

According to a more preferred embodiment, the invention provides a process for preparing a pharmaceutical composition in the form of an injectable dosage form, comprising:
a) preparing a solution by adding approximately 30 mg to 40 mg of sodium hydroxide in water for injection;
b) adding and dissolving approximately 530 mg to 550 mg of Chlorothiazide sodium or polymorphs thereof, into the solution of step a) to obtain Solution A;
c) adding and dissolving approximately 225 mg to 275 mg of mannitol into the Solution A to obtain Solution B;
d) optionally adjusting the pH of Solution B between 9.0 and 10.0 by adding a pH adjusting agent to obtain Solution C;
e) optionally adjusting the volume of the Solution B or Solution C by adding additional water for injection;
f) filtering the Solution B or Solution C or solution from step e) through a membrane filter;
g) filling said filtered solution from step f) into a vial; and
h) optionally lyophilizing said solution from step g) to obtain a white lyophilized product.
   According to one embodiment, the process for the preparation of a parenteral pharmaceutical composition comprising Chlorothiazide or a pharmaceutically acceptable salt thereof, in particular Chlorothiazide sodium, said process comprising,
a) preparing a dispersion of Chlorothiazide free base in a pharmaceutically acceptable vehicle;
b) preparing a solution of sodium hydroxide in a pharmaceutically acceptable vehicle;
c) converting in situ the Chlorothiazide free base into Chlorothiazide sodium by addition of sodium hydroxide solution into Chlorothiazide dispersion of step a) to obtain Solution A;
d) adding and dissolving the tonicity agent into Solution A to obtain Solution B;
e) optionally adjusting the pH of Solution B between 9.0 and 10.0 by adding a pH adjusting agent to obtain Solution C;
f) optionally adjusting the volume of the Solution B or Solution C by adding additional pharmaceutically acceptable vehicle;
g) filtering the Solution B or Solution C or solution from step f) through a membrane filter;
h) filling the filtered solution into a vial; and
i) optionally lyophilizing said solution from step h) to obtain a white lyophilized product.

According to a preferred embodiment, the process for the preparation of a parenteral pharmaceutical composition comprising Chlorothiazide or a pharmaceutically acceptable salt thereof, in particular Chlorothiazide sodium, said process comprising,
a) preparing a dispersion of Chlorothiazide free base in a pharmaceutically acceptable vehicle;
b) preparing a solution of sodium hydroxide in a pharmaceutically acceptable vehicle;
c) converting in situ the Chlorothiazide free base into Chlorothiazide sodium by slow addition of sodium hydroxide solution into Chlorothiazide dispersion of step a) to obtain Solution A;
d) adding and dissolving the tonicity agent into Solution A to obtain Solution B;
e) optionally adjusting the pH of Solution B between 9.0 and 10.0 by adding a pH adjusting agent to obtain Solution C;
f) optionally adjusting the volume of the Solution B or Solution C by adding additional pharmaceutically acceptable vehicle;
g) filtering the Solution B or Solution C or solution from step f) through a membrane filter;
h) filling the filtered solution into a vial; and
i) optionally lyophilizing the solution from step h) to obtain a white lyophilized product.

According to another preferred embodiment, the process for the preparation of a parenteral pharmaceutical composition comprising Chlorothiazide or a pharmaceutically acceptable salt thereof, in particular Chlorothiazide sodium, said process comprising,
a) preparing a dispersion of Chlorothiazide free base in a pharmaceutically acceptable vehicle;
b) preparing a solution of sodium hydroxide in a pharmaceutically acceptable vehicle;
c) converting in situ the Chlorothiazide free base into Chlorothiazide sodium by fast addition of sodium hydroxide solution into Chlorothiazide dispersion of step a) to obtain Solution A;
d) adding and dissolving the tonicity agent into Solution A to obtain Solution B;
e) optionally adjusting the pH of the Solution B between 9.0 and 10.0 by adding a pH adjusting agent to obtain Solution C;
f) optionally adjusting the volume of the Solution B or Solution C by adding additional pharmaceutically acceptable vehicle;
g) filtering the Solution B or Solution C or solution from step f) through a membrane filter;
h) filling the filtered solution into a vial; and
i) optionally lyophilizing the solution from step h) to obtain a white lyophilized product.

According to a preferred embodiment, the process for the preparation of a parenteral pharmaceutical composition comprising Chlorothiazide or a pharmaceutically acceptable salt thereof, in particular Chlorothiazide sodium, said process comprising
a) preparing a dispersion of Chlorothiazide free base in water for injection;
b) preparing a solution of sodium hydroxide in water for injection;
c) converting in situ the Chlorothiazide free base into Chlorothiazide sodium by slow addition of sodium hydroxide solution into Chlorothiazide dispersion of step a) to obtain Solution A;
d) adding and dissolving mannitol into the Solution A to obtain Solution B;
e) optionally adjusting the pH of the Solution B between 9.0 and 10.0 by adding sodium hydroxide solution to obtain Solution C;
f) optionally adjusting the volume of the Solution B or Solution C by adding additional water for injection;
g) filtering the Solution B or Solution C or solution from step through a membrane filter;
h) filling the filtered solution into a vial; and
i) optionally lyophilizing the solution from step h) to obtain a white lyophilized product.

According to a preferred embodiment, the process for the preparation of a parenteral pharmaceutical composition comprising Chlorothiazide or a pharmaceutically acceptable salt thereof, in particular Chlorothiazide sodium, said process comprising,
a) preparing a dispersion of Chlorothiazide free base in water for injection;
b) preparing a solution of sodium hydroxide in water for injection;
c) converting in situ the Chlorothiazide free base into Chlorothiazide sodium by fast addition of sodium hydroxide solution into Chlorothiazide dispersion of step a) to obtain Solution A;
d) adding and dissolving mannitol into the Solution A to obtain Solution B;
e) optionally adjusting the pH of the Solution B between 9.0 and 10.0 by adding sodium hydroxide solution to obtain Solution C;
f) optionally adjusting the volume of the Solution B or Solution C by adding additional water for injection;
g) filtering the Solution B or Solution C or solution from step f) through a membrane filter;
h) filling the filtered solution into a vial; and
i) optionally lyophilizing the solution from step h) to obtain a white lyophilized product.

According to a more preferred embodiment, the invention provides a process for preparing a parenteral pharmaceutical composition comprising Chlorothiazide sodium, said process comprising,
a) preparing a dispersion by adding approximately 275 mg to 550 mg Chlorothiazide sodium or polymorph thereof, in pharmaceutically acceptable vehicle/carrier;
b) adding a solution of pH adjusting agent (25 mg-45 mg of pH adjusting agent dissolved in pharmaceutically acceptable vehicle) into the dispersion of step a) to obtain Solution A;
c) adding and dissolving approximately 150 mg to 350 mg of tonicity agent into Solution A to obtain Solution B;
d) optionally adjusting the pH of the Solution B between 9.0 and 10.0 by adding a pH adjusting agent to obtain Solution C;
e) optionally adjusting the volume of Solution B or Solution C by adding additional pharmaceutically acceptable vehicle;
f) filtering Solution B or Solution C or solution from step e) through a membrane filter;
g) filling said filtered solution from step f) into a vial;
h) optionally lyophilizing said solution from step g) to obtain a white lyophilized product.

Pharmaceutical compositions according to the present invention are found to be stable. Long term stability studies are under progress.

Isotonic solutions possess the same osmotic pressure as blood plasma and hence can be intravenously infused into a subject without changing the osmotic pressure of the subject's blood. In the practice of the present invention, the tonicity agent that may be used includes but are not limited to sodium chloride, dextrose, mannitol, sucrose, lactose, galactose, trehalose, glycine or mixture thereof. In the practice of the present invention, the pH adjusting agent that may be used includes but are not limited to sodium hydroxide, potassium hydroxide, alkali or alkaline earth metal carbonates/bicarbonates or mixture thereof.

The pharmaceutically acceptable vehicle of the present invention optionally comprises buffers, antioxidants or chelating agents. Examples of buffers that may be used according to the invention include but are not limited to phosphates, citrates, acetates, carbonate, borates and mixture thereof. Examples of antioxidants that may be used according to the invention include but are not limited to ascorbic acid, citric acid, sodium citrate, sodium metabisulfite, malic acid, butylated hydroxyanisole, butylated hydroxytoluene and mixture thereof.

The injectable compositions of the present invention may be dispensed in containers such as in ampoules, vials, prefilled syringes. The pharmaceutical compositions of the present invention may be sterilized by any of the sterilizing methods such as sterilization by filtration, autoclaving, aseptic sterilization or a combination of any of the methods. In the practice of the present invention, the composition of the present invention may be sterilized using membrane filters having pore size of about 0.45 microns to 0.2 microns. Suitable membrane filters that may be used include Polyvinylidene fluoride (PVDF), Polyethersulfone (PES), cellulose acetate membrane, nitrocellulose and the like.

According to one embodiment, the present invention provides a method of treating a patient suffering from hypertension and edema associated with congestive heart failure, comprising administering to a patient in need thereof a therapeutically effective amount of Chlorothiazide sodium in pharmaceutical composition as described herein.

According to another embodiment, the Chlorothiazide sodium as described herein can be used in the preparation of a medicament for treating a mammal suffering from hypertension and edema associated with congestive heart failure.

X-ray powder diffraction pattern are obtained on Xpert'PRO, PANalytical, diffractometer equipped with accelerator detector using Copper Kα (n=1.5406 \acute{Å}) radiation with scanning range between 2-theta 4-50° at a scanning speed of 2°/min.

The following examples are for illustrative purposes only and are not intended to limit the scope of the invention. The invention is intended to cover all alternatives, modifications and equivalents included within its spirit and scope.

### EXAMPLES

### Example 1

100 g of 5-chloro-2,4-disulfamylaniline was suspended in 500 ml of about 98% formic acid. The obtained suspension was heated at a temperature of about 90-100° C. The solid was precipitated out from the clear suspension at 90-92° C. The suspension was maintained at the same temperature for 5 hours. The hot suspension was cooled to 25-30° C. and the obtained solid was filtered followed by washing with acetone to get 94 gm of Chlorothiazide.

### Example 2

### Purification of Chlorothiazide

90 g of Chlorothiazide was suspended in 900 ml acetone. The suspension was heated to reflux and at reflux temperature, 270 ml water was added to it to get a clear solution. The solution was cooled to a temperature of 0 to −5° C. 270 ml of water was added to the cooled solution for precipitating out the solid. The suspension was maintained at this temperature for 2 hrs and the solid was isolated by filtration followed by washing with acetone to get 55 g of pure Chlorothiazide.

Purity: more than 99.5%.

### Example 3

### Preparation of Chlorothiazide Sodium

7 g of sodium hydroxide was dissolved in 250 ml ethanol at 60-65° C. to get clear solution. 50 g of pure Chlorothiazide was added at the same temperature to the obtained clear solution of sodium hydroxide. The obtained suspension was stirred at same temperature for 30 min., cooled to 25-30° C. and maintained for 2-3 hrs. The separated solid was filtered, washed with ethanol and dried at 75-80° C. to get 45 g of Chlorothiazide sodium.

### Preparation of Chlorothiazide Form I

### Example 4

5 g of crude Chlorothiazide was suspended in 50 ml methanol at 25-30° C. The suspension was heated to reflux. 15 ml water was added dropwise to the heated suspension at reflux temperature to get a clear solution. The clear solution was cooled to 0 to 5° C. 15 ml water was added to it and maintained at the same temperature for 2 hrs. The separated solid was filtered and dried to get 3.5 g of pure Chlorothiazide Form I.

Purity: more than 99%.

### Example 5

5 g of crude Chlorothiazide was suspended in 50 ml ethanol at 25-30° C. The suspension was heated to reflux. 15 ml water was added dropwise to the heated suspension at reflux temperature to get a clear solution. The clear solution was cooled to 0 to 5° C. 15 ml water was added to it and maintained at the same temperature for 2 hrs. The separated solid was filtered and dried to get 3.25 g pure Chlorothiazide Form I. Purity: more than 99%.

### Example 6

5 g of crude Chlorothiazide was suspended in 50 ml isopropanol at 25-30° C. The suspension was heated to reflux. 15 ml water was added dropwise to the heated suspension at reflux temperature to get a clear solution. The clear solution was cooled to 0 to 5° C. 15 ml water was added to it and maintained at the same temperature for 2 hrs. The separated solid was filtered and dried to get pure 3.5 g Chlorothiazide Form I. Purity: more than 99%.

### Example 7

5 g of crude Chlorothiazide was suspended in 50 ml acetone at 25-30° C. The suspension was heated to reflux. 15 ml water was added dropwise to the heated suspension at reflux temperature to get a clear solution. The clear solution was cooled to 0 to 5° C. 15 ml water was added to it and maintained at the same temperature for 2 hrs. The separated solid was filtered and dried to get pure 3.6 g Chlorothiazide Form I. Purity: more than 99.5%.

### Example 8

5 g of crude Chlorothiazide was suspended in 50 ml acetonitrile at 25-30° C. The suspension was heated to reflux. 15 ml water was added dropwise to the heated suspension at reflux temperature to get a clear solution. The clear solution was cooled to 0 to 5° C. 15 ml water was added to it and maintained at the same temperature for 2 hrs. The separated solid was filtered and dried to get 3.4 g pure Chlorothiazide Form I. Purity: more than 99%.

### Example 9

5 g of crude Chlorothiazide was suspended in 50 ml tetrahydrofuran at 25-30° C. The suspension was heated to reflux. 15 ml water was added dropwise to the heated suspension at reflux temperature to get a clear solution. The clear solution was cooled to 0 to 5° C. 15 ml water was added to it and maintained at the same temperature for 2 hrs. The separated solid was filtered and dried to get 3.85 g pure Chlorothiazide Form I. Purity: more than 99%.

### Example 10

5 g of crude Chlorothiazide was suspended in 50 ml 1,4-dioxane at 25-30° C. The suspension was heated to reflux. 15 ml water was added dropwise to the heated suspension at reflux temperature to get a clear solution. The clear solution was cooled to 0 to 5° C. 15 ml water was added to it and maintained at the same temperature for 2 hrs. The separated solid was filtered and dried to get 2.8 g pure Chlorothiazide Form I. Purity: more than 99%.

### Example 11

5 g of crude Chlorothiazide was dissolved in 1.5 ml of DMF at 25-30° C. The obtained solution was filtered to remove any suspended particles and poured over 25 ml of ice cold water. The resulting mixture was stirred for 1 hr. The solid was isolated by filtration and dried to get 3.33 g pure Chlorothiazide Form I.

Purity: more than 99%.

### Example 12

5 g of crude Chlorothiazide was dissolved in 1.5 ml of DMSO at 25-30° C. The obtained solution was filtered to remove any suspended particles and poured over 25 ml of ice cold water. The resulting mixture was stirred for 1 hr. The solid was isolated by filtration and dried to get 2.75 g pure Chlorothiazide Form I.

Purity: more than 99%.

### Example 13

5 g of crude Chlorothiazide was dissolved in 1.5 ml of DMF at 25-30° C. The obtained solution was filtered to remove any suspended particles. The solution was poured over 25 ml of toluene at reflux and stirred for 1 hr. The solid was isolated by filtration and dried to get 2.36 g pure Chlorothiazide Form I. Purity: more than 99%.

### Example 14

0.5 g of crude Chlorothiazide was dissolved in 1.5 ml DMSO at 25-30° C. The solution was filtered to remove any suspended particles. The solution was poured over 25 ml toluene at reflux and stirred for 1 hr. The solid was separated by filtration and dried to get 2.64 g pure Chlorothiazide Form I. Purity: more than 99%.

### Example 15

2 g of crude Chlorothiazide was dissolved in 2 ml DMF at 25-30° C. 5 ml acetone was added to the obtained solution. The solution was filtered to remove any suspended particles. 10 ml MTBE was added to the clear solution. The resulting mixture was stirred for 1 hr. The solid was isolated by filtration and dried to get 1.4 g pure Chlorothiazide Form I. Purity: more than 99%.

### Preparation of Chlorothiazide Form II

### Example 16

1 g of Chlorothiazide was dissolved in 10 ml DMF at 25-30° C. The solution was filtered to get a clear solution. The obtained clear solution was evaporated under vacuum at 80° C. to get 0.45 g Chlorothiazide Form II.

### Preparation of Chlorothiazide Sodium Form I

### Example 17

0.7 g of sodium hydroxide was dissolved in 100 ml methanol at 50-55° C. to get clear solution. 5 g of Chlorothiazide was added to the sodium hydroxide solution at the same temperature. The obtained solution was concentrated under vacuum at 50° C. The separated solid was isolated and dried at 75-80° C. to get 4.6 g of Chlorothiazide sodium Form I having X-ray diffraction pattern as shown in FIG. 4.

### Example 18

0.7 g of sodium hydroxide was dissolved in 50 ml methanol at 50-55° C. to get clear solution. 5 g of Chlorothiazide was added to the sodium hydroxide solution at the same temperature. The obtained suspension was stirred for 2 hrs. The separated solid was isolated and dried at 75-80° C. to get 4.2 g of substantially pure Chlorothiazide sodium Form I having X-ray diffraction pattern as shown in FIG. 5.

Purity: more than 99.5%.

### Example 19

0.7 g of sodium hydroxide was dissolved in 50 ml methanol at 50-55° C. to get clear solution. 5 g of Chlorothiazide was added to the sodium hydroxide solution at the same temperature. The obtained solution was poured in 50 ml MTBE and stirred for 1 hr. The separated solid was isolated by filtration and dried at 75-80° C. to get 4.35 g of substantially pure Chlorothiazide sodium Form I having X-ray diffraction pattern as shown in FIG. 5. Purity: more than 99.5%.

### Example 20

0.7 g of sodium hydroxide was dissolved in 50 ml methanol at 50-55° C. to get clear solution. 5 g of Chlorothiazide was added to the sodium hydroxide solution at the same temperature. The obtained solution was poured in 50 ml diisopropyl ether (DIPE) and stirred for 1 hr. The separated solid was isolated by filtration and dried at 75-80° C. to get 4.6 g of substantially pure Chlorothiazide sodium Form I having X-ray diffraction pattern as shown in FIG. 5. Purity: more than 99.5%.

### Example 21

0.7 g of sodium hydroxide was dissolved in 50 ml methanol at 50-55° C. to get clear solution. 5 g of Chlorothiazide was added to the sodium hydroxide solution at the same temperature. The obtained solution was poured in 50 ml toluene and stirred for 1 hr. The separated solid was isolated by filtration and dried at 75-80° C. to get 4.35 g of substantially pure Chlorothiazide sodium Form I having X-ray diffraction pattern as shown in FIG. 5. Purity: more than 99.5%.

### Example 22

0.7 g of sodium hydroxide was dissolved in 50 ml methanol at 50-55° C. to get clear solution and 5 g of Chlorothiazide was added to the sodium hydroxide solution at the same temperature. The obtained solution was poured in 60 ml acetonitrile and stirred for 1 hr. The separated solid was isolated by filtration and dried at 75-80° C. to get 4.15 g of substantially pure Chlorothiazide sodium Form I having X-ray diffraction pattern as shown in FIG. 5. Purity: more than 99.5%.

### Example 23

0.7 g of sodium hydroxide was dissolved in 50 ml methanol at 50-55° C. to get clear solution. 5 g of Chlorothiazide was added to the sodium hydroxide solution at the same temperature. The obtained solution was poured in 50 ml dichloromethane and stirred for 1 hr. The separated solid was isolated by filtration and dried at 75-80° C. to get 4.25 g of substantially pure Chlorothiazide sodium Form I having X-ray diffraction pattern as shown in FIG. 5. Purity: more than 99.5%.

Preparation of Chlorothiazide sodium Form II.

### Example 24

0.7 g of sodium hydroxide was dissolved in 50 ml ethanol at 50-55° C. to get clear solution. 5 g of Chlorothiazide was added to the clear solution at the same temperature. The obtained solution was stirred for 1 hr. The separated solid was isolated by filtration and dried immediately under vacuum at 75-80° C. to get 4.55 g of Chlorothiazide sodium Form II. Purity: more than 99.5%.

### Example 25

0.9 g of sodium methoxide was dissolved in 50 ml ethanol at 50-55° C. to get clear solution. 5 g of Chlorothiazide was added to the clear solution at the same temperature. The obtained solution was stirred for 1 hr. The separated solid was isolated by filtration and dried immediately under vacuum at 75-80° C. to get 4.55 g of Chlorothiazide sodium Form II. Purity: more than 99.5%.

### Preparation of Chlorothiazide Sodium Form III

### Example 26

0.9 g of sodium methoxide was dissolved in 50 ml methanol at 50-55° C. to get clear solution. 5 g of Chlorothiazide was added to the clear solution at the same temperature. The obtained solution was stirred for 1 hr. The separated solid was isolated by filtration and dried at 75-80° C. to get 4.55 g Chlorothiazide sodium Form III. Purity: more than 99.5%.

### Preparation of Chlorothiazide Sodium Form IV

### Example 27

4 g of Chlorothiazide sodium was dried under vacuum at 100-120° C. for 24 hrs to get 3.6 g of Chlorothiazide sodium Form IV.

### Preparation of Chlorothiazide Sodium Composition (Parenteral)

### Example 28

| Ingredient | Quantity (mg/Vial) |
|---|---|
| Chlorothiazide sodium Form II | 539.02 |
| | (equivalent to 500 mg |
| | of Chlorothiazide) |
| Mannitol | 250.00 |
| Sodium Hydroxide | 35.40 |
| Sodium Hydroxide, 1% (w/v) Solution | q.s. |

35.40 mg of sodium hydroxide was added to water for injection (70% of the batch size) with constant stirring at 300-400 rpm to get a clear solution. The solution was maintained at a temperature of about 5° C. to 15° C. 539.02 mg of Chlorothiazide sodium Form II was added to this solution with constant stirring. 250 mg of mannitol was added to this solution with stirring. The pH of the solution was adjusted to 9.2 using 1% sodium hydroxide solution. The solution was subjected to nitrogen sparging. The volume was made up to 10 ml with water for injection. The solution was filtered using membrane filter and filled into vials and partially stoppered. The vials were lyophilized and the lyophilized vials were finally stoppered and sealed with aluminium caps.

### Example 29

| Ingredient | Quantity (mg/Vial) |
|---|---|
| Chlorothiazide sodium Form II | 539.02 |
| | (equivalent to 500 mg |
| | of Chlorothiazide) |
| Mannitol | 250.00 |
| Sodium Hydroxide | 35.40 |
| Sodium Hydroxide, 1% (w/v) Solution | q.s. |

539.02 mg of Chlorothiazide sodium Form II was added to water for injection (80% of the batch size) with constant stirring at 300-400 rpm to get a dispersion. Sodium hydroxide solution (prepared by dissolving 35.40 mg of sodium hydroxide in water for injection) was added slowly to this dispersion with constant stirring. The solution was maintained at a temperature of about 5° C. to 15° C. 250 mg of mannitol was added to this solution with stirring. The pH of the solution was adjusted to 9.2 using 1% sodium hydroxide solution. The solution was subjected to nitrogen sparging. The volume was made up to 10 ml with water for injection. The solution was filtered using membrane filter and filled into vials and partially stoppered. The vials were lyophilized and the lyophilized vials were finally stoppered and sealed with aluminium caps.

### Example 30

| Ingredient | Quantity (mg/Vial) |
|---|---|
| Chlorothiazide sodium Form II | 539.02 |
| | (equivalent to 500 mg |
| | of Chlorothiazide) |
| Sucrose | 200.00 |
| Potassium Hydroxide | 30.00 |
| Potassium Hydroxide 1% (w/v) Solution | q.s. |

30 mg of potassium hydroxide was added to water for injection (80% of the batch size) with constant stirring at 300-400 rpm to get a clear solution. The solution was maintained at a temperature of about 5° C. to 15° C. 539.02 mg of Chlorothiazide sodium Form II was added to this solution with constant stirring. 200 mg of sucrose was added to this solution with stirring. The pH of the solution was adjusted between 9.0 and 10.0 using 1% potassium hydroxide solution. The solution was subjected to nitrogen sparging. The volume was made up to 10 ml with water for injection. The solution was filtered using membrane filter and filled into vials and partially stoppered. The vials were lyophilized and the lyophilized vials were finally stoppered and sealed with aluminium caps.

### Example 31

| Ingredient | Quantity (mg/Vial) |
|---|---|
| Chlorothiazide base Form I | 500.00 |
| Mannitol | 250.00 |
| Sodium Hydroxide | 96.00 |
| Sodium Hydroxide, 1% (w/v) Solution | q.s. |

Chlorothiazide base Form I 500 mg was added to water for injection (70% of the batch size) with constant stirring at 300-400 rpm to get a dispersion. 96 mg of sodium hydroxide was added to water for injection (30% of the batch size) with constant stirring at 300-400 rpm to get a clear solution. This sodium hydroxide solution was slowly added to the dispersion of Chlorothiazide base with constant stirring to get a solution. This solution was maintained at a temperature of about 5° C. to 15° C. 250 mg of mannitol was added to this solution with stirring. The pH of the solution was adjusted to 9.5 using 1% sodium hydroxide solution. The solution was subjected to nitrogen sparging. The volume was made up to 10 ml with water for injection. The solution was filtered using membrane filter and filled into vials and partially stoppered. The vials were lyophilized and the lyophilized vials were finally stoppered and sealed with aluminium caps.

### Example 32

| Ingredient | Quantity (mg/Vial) |
|---|---|
| Chlorothiazide base Form I | 500.00 |
| Mannitol | 250.00 |
| Sodium Hydroxide | 96.00 |
| Sodium Hydroxide, 1% (w/v) Solution | q.s. |

Chlorothiazide base Form I 500 mg was added to water for injection (70% of the batch size) with constant stirring at 300-400 rpm to get a dispersion. 96 mg of sodium hydroxide was added to water for injection (30% of the batch size) with constant stirring at 300-400 rpm to get a clear solution. This sodium hydroxide solution was added to the dispersion of Chlorothiazide base by fast addition method with constant stirring to get a solution. This solution was maintained at a temperature of about 5° C. to 15° C. 250 mg of mannitol was added to this solution with stirring. The pH of the solution was adjusted to 9.5 using 1% sodium hydroxide solution. The solution was subjected to nitrogen sparging. The volume was made up to 10 ml with water for injection. The solution was filtered using membrane filter and filled into vials and partially stoppered. The vials were lyophilized and the lyophilized vials were finally stoppered and sealed with aluminium caps.

### Example 33

| Ingredient | Quantity (mg/Vial) |
|---|---|
| Chlorothiazide base Form I | 500.00 |
| Mannitol | 250.00 |
| Sodium Hydroxide | 96.00 |
| Sodium Hydroxide, 1% (w/v) Solution | q.s. |

96.00 mg of sodium hydroxide was added to water for injection (70% of the batch size) with constant stirring at 300-400 rpm to get a clear solution. The solution was maintained at a temperature of about 5° C. to 15° C. 500 mg of Chlorothiazide base was added to this solution with constant stirring. 250 mg of mannitol was added to this solution with stirring. The pH of the solution was adjusted between 9.0 to 10.0 using 1% sodium hydroxide solution. The solution was subjected to nitrogen sparging. The volume was made up to 10 ml with water for injection. The solution was filtered using membrane filter and filled into vials and partially stoppered. The vials were lyophilized and the lyophilized vials were finally stoppered and sealed with aluminium caps.

### Example 34

| Ingredient | Quantity (mg/Vial) |
|---|---|
| Chlorothiazide sodium Form II | 539.02 |
| | (equivalent to 500 mg |
| | of Chlorothiazide) |
| Mannitol | 250.00 |
| Sodium Hydroxide | 35.40 |
| Sodium Hydroxide, 1% (w/v) Solution | q.s. |

539.02 mg of Chlorothiazide sodium Form II was added to water for injection (80% of the batch size) with constant stirring at 300-400 rpm to get a clear solution. Sodium hydroxide solution (prepared by dissolving 35.40 mg of sodium hydroxide in water for injection) was added to this dispersion by fast addition method with constant stirring. The solution was maintained at a temperature of about 5° C. to 15° C. 250 mg of mannitol was added to this solution with stirring. The pH of the solution was adjusted between 9.0 and 10.0 using 1% sodium hydroxide solution. The solution was subjected to nitrogen sparging. The volume was made up to 10 ml with water for injection. The solution was filtered using membrane filter and filled into vials and partially stoppered. The vials were lyophilized and the lyophilized vials were finally stoppered and sealed with aluminium caps.

We claim:

1. A process for preparation of Chlorothiazide or pharmaceutically acceptable salts thereof comprising,
a) treating 5-chloro-2,4-disulfamylaniline with 5 volumes of formic acid to obtain Chlorothiazide;
b) optionally purifying Chlorothiazide; and
c) optionally converting Chlorothiazide to its pharmaceutically acceptable salts.

2. The process as claimed in claim 1, wherein said treatment in step a) is carried out at a temperature of 80-100° C. for about 4 to 6 hours.

3. The process as claimed in claim 1, wherein process of purification of Chlorothiazide comprises,
a) treating Chlorothiazide with a first solvent at a temperature of about 25-100° C. or at reflux temperature to obtain a suspension;
b) optionally adding second solvent to the suspension formed in step (a); and
c) isolating purified Chlorothiazide.

4. The process as claimed in claim 3, wherein said isolated purified Chlorothiazide is Chlorothiazide Form I characterized by X-ray diffraction pattern having peaks at 2-theta values of about 14.42, 19.81, 20.51, 21.80, 26.29, 28.20, 28.67 and 29.10 deg.

5. The process as claimed in claim 3, wherein said first solvent is selected from methanol, ethanol, isopropanol, acetone, acetonitrile, dimethyl formamide, dimethyl sulfoxide, tetrahydrofuran or 1,4-dioxane; and wherein second solvent is selected from water, diethyl ether, diisopropyl ether, methyl tertbutyl ether, hexane, heptane or toluene.

6. The process as claimed in claim 1, wherein said Chlorothiazide obtained in step a) or step b) is treated with a polar aprotic solvent selected from dimethylformamide or dimethyl sulfoxide followed by isolation of Chlorothiazide Form II characterized by X-ray diffraction pattern having peaks at 2-theta values of about 8.13, 17.02, 18.55, 19.38, 20.62, 20.95, 21.96, 26.03, 27.76 and 32.90 deg.

7. The process as claimed in claim 1, wherein the pharmaceutically acceptable salt is Chlorothiazide sodium.

8. The process as claimed in claim 7, wherein said Chlorothiazide is converted to Chlorothiazide sodium by a process comprising the steps of,
a) treating Chlorothiazide with a suitable alkaline medium to get a solution;
b) optionally, adding antisolvent to the solution obtained in step (a); and
c) isolating Chlorothiazide sodium.

9. The process as claimed in claim 8, wherein suitable alkaline medium is selected from sodium hydroxide, sodium methoxide or sodium ethoxide in a suitable solvent selected from water, methanol, ethanol, n-propanol, isopropanol or mixture thereof; and wherein antisolvent is selected from hexane, heptane, toluene, methylene dichloride, diethyl ether, diisopropyl ether, methyl tertbutyl ether, tetrahydrofuran, 1,4-dioxane, acetonitrile, acetone, ethyl methyl ketone, 2-butanone or mixture thereof.

10. The process as claimed in claim 8, wherein said Chlorothiazide sodium is substantially pure Chlorothiazide sodium Form I

11. The process as claimed in claim 8, wherein said isolated Chlorothiazide sodium is dried immediately under vacuum to obtain Chlorothiazide sodium Form II characterized by X-ray diffraction pattern having peaks at 2-theta values of about 17.22, 19.82, 25.91, 27.60, 29.55 and 31.21 deg.

12. The process as claimed in claim 8, wherein said Chlorothiazide is selected from Chlorothiazide Form I or Form II.

13. The process as claimed in claim 8, wherein said isolated Chlorothiazide sodium is Chlorothiazide sodium Form III characterized by X-ray diffraction pattern having peaks at 2-theta values of about 8.83, 10.41, 16.70, 18.15, 20.62, 23.31, 26.38, 26.59 and 28.11 deg.

14. The process as claimed in claim 8, wherein said isolated Chlorothiazide sodium is subjected to drying at a temperature of about 100 to 120° C. for 8-12 hours to get Chlorothiazide sodium Form IV characterized by X-ray diffraction pattern having peaks at 2-theta values of about 16.55, 17.12, 17.56, 18.09, 19.55, 19.72 and 27.52 deg.

15. The process as claimed in claim 14, wherein said Chlorothiazide sodium subjected to drying is Chlorothiazide sodium Form I, Form II or Form III.

16. A compound selected from the group consisting of:
a) Chlorothiazide Form II characterized by X-ray diffraction pattern having peaks at 2-theta values of about 8.13, 17.02, 18.55, 19.38, 20.62, 20.95, 21.96, 26.03, 27.76 and 32.90 deg;
b) Chlorothiazide sodium Form II characterized by X-ray diffraction pattern having peaks at 2-theta values of about 17.22, 19.82, 25.91, 27.60, 29.55 and 31.21 deg;
c) Chlorothiazide sodium Form III characterized by X-ray diffraction pattern having peaks at 2-theta values of about 8.83, 10.41, 16.70, 18.15, 20.62, 23.31, 26.38, 26.59 and 28.11; and
d) Chlorothiazide sodium Form IV characterized by X-ray diffraction pattern having peaks at 2-theta values of about 16.55, 17.12, 17.56, 18.09, 19.55, 19.72 and 27.52.

17. A parenteral pharmaceutical composition comprising:
a) a therapeutically effective amount of Chlorothiazide, Chlorothiazide sodium or polymorphic form thereof prepared by the process as claimed in any of the preceding claims;
b) a pharmaceutically acceptable tonicity agent; and
c) a pharmaceutically acceptable pH adjusting agent.

18. A parenteral pharmaceutical composition comprising:
a) a therapeutically effective amount of Chlorothiazide sodium;
b) a pharmaceutically acceptable tonicity agent; and
c) a pharmaceutically acceptable pH adjusting agent;
wherein said Chlorothiazide sodium is formed by the in situ conversion of Chlorothiazide free base into Chlorothiazide sodium.

19. The composition as claimed in claim 18 wherein said Chlorothiazide free base is Chlorothiazide Form I

20. A parenteral pharmaceutical composition comprising:
a) a therapeutically effective amount of Chlorothiazide sodium or polymorphs thereof, preferably Chlorothiazide sodium Form II or substantially pure Form I;
b) a pharmaceutically acceptable tonicity agent; and
c) a pharmaceutically acceptable pH adjusting agent.

21. The composition as claimed in any of the preceding claims wherein said pH adjusting agent is selected from the group consisting of sodium hydroxide, potassium hydroxide, alkali or alkaline earth metal carbonates/bicarbonates or mixture thereof; and said tonicity agent is selected from sodium chloride, dextrose, mannitol, sucrose, lactose, galactose, trehalose, glycine or mixture thereof.

22. A process for the preparation of parenteral pharmaceutical composition comprising Chlorothiazide sodium comprising the steps of:
a) preparing a dispersion of Chlorothiazide free base in a pharmaceutically acceptable vehicle;
b) converting in situ said Chlorothiazide free base into Chlorothiazide sodium by adding sodium hydroxide solution to form Solution A;
c) adding and dissolving tonicity agent into Solution A to obtain Solution B;
d) optionally adjusting pH of Solution B between 9.0 and 10.0 by adding pH adjusting agent; and
e) optionally lyophilizing said Solution B to obtain a white lyophilized product.

23. The process for preparation of parenteral composition as claimed in claim 22, said process comprising:
10. preparing a dispersion of Chlorothiazide free base Form I in a pharmaceutically acceptable vehicle;
11. preparing a solution of sodium hydroxide in a pharmaceutically acceptable vehicle;
12. converting in situ said Chlorothiazide free base Form I into Chlorothiazide sodium by addition of sodium hydroxide solution into Chlorothiazide dispersion of step a) to obtain Solution A;
13. adding and dissolving the tonicity agent into said Solution A to obtain Solution B;
14. optionally adjusting the pH of said Solution B between 9.0 and 10.0 by adding a pH adjusting agent to obtain Solution C;
15. optionally adjusting the volume of said Solution B or Solution C by adding additional pharmaceutically acceptable vehicle;
16. filtering said Solution B or Solution C or solution from step f) through a membrane filter;
17. filling said filtered solution into a vial; and
18. optionally lyophilizing said solution from step h) to obtain a white lyophilized product.

24. The process for the preparation of parenteral composition as claimed in claim 23, said process comprising:
a) preparing a dispersion of Chlorothiazide free base Form I in water for injection;
b) preparing a solution of sodium hydroxide in water for injection;
c) converting in situ said Chlorothiazide free base Form I into Chlorothiazide sodium by addition of sodium hydroxide solution into Chlorothiazide dispersion of step a) to obtain Solution A;
d) adding and dissolving mannitol into said Solution A to obtain Solution B;
e) optionally adjusting the pH of Solution B between 9.0 and 10.0 by adding sodium hydroxide solution to obtain Solution C;
f) optionally adjusting the volume of said Solution B or Solution C by adding additional water for injection;
g) filtering said Solution B or Solution C or solution from step f) through a membrane filter;
h) filling said filtered solution into a vial; and
i) optionally lyophilizing said solution from step h) to obtain a white lyophilized product.

25. The process as claimed in any of the claims 22 to 24 wherein said addition of sodium hydroxide solution into Chlorothiazide dispersion is carried out either by slow addition method or fast addition method.

26. A process for the preparation of parenteral pharmaceutical composition comprising Chlorothiazide sodium, said process comprising:
i) preparing a solution by adding pH adjusting agent in a pharmaceutically acceptable vehicle;
j) adding and dissolving Chlorothiazide sodium or polymorphs thereof, into the solution of step (a) to obtain Solution A;
k) adding and dissolving tonicity agent into said Solution A to obtain Solution B;
l) optionally adjusting the pH of said Solution B between 9.0 and 10.0 by adding a pH adjusting agent to obtain Solution C;
m) optionally adjusting the volume of said Solution B or Solution C by adding additional pharmaceutically acceptable vehicle;
n) filtering said Solution B or Solution C or solution from step e) through a membrane filter;
o) filling said filtered solution into a vial; and
p) optionally lyophilizing said solution from step g) to obtain a white lyophilized product.

27. The process as claimed in any of the preceding claims 22 to 26 wherein a nitrogen sparge is optionally used to reduce the dissolved oxygen.

28. The process as claimed in any of the preceding claims 22 to 27 wherein said pH adjusting agent is selected from the group consisting of sodium hydroxide, potassium hydroxide, alkali or alkaline earth metal carbonates/bicarbonates or mixture thereof; and said tonicity agent is selected from sodium chloride, dextrose, mannitol, sucrose, lactose, galactose, trehalose, glycine or mixture thereof.

29. The process as claimed in claim 28 wherein Chlorothiazide is used in the range of about 1% to 20% by weight of total composition; tonicity agent is used in the range of about 1% to 15% by weight of total composition; and pH adjusting agent is used in the range of about 0.1% to 5% by weight of total composition.

30. The composition as claimed in any of the preceding claims, wherein said composition is used for treating a mammal suffering from hypertension and edema associated with congestive heart failure.
